(19) United States
(12) Patent Application Publication
(10) Pub. No.: US 2026/0283591 A1
(43) Pub. Date: Sep. 24, 2026

(54) SYSTEM AND METHOD FOR WHITE BLOOD CELL COUNTING IN SEROUS BODY FLUIDS

(71) Applicant: NEOS NEW BORN SOLUTIONS, S.L., Madrid (ES)

(72) Inventors: Javier JIMÉNEZ GONZÁLEZ, Madrid (ES); Ramon SERRANO DELOUNDER, Madrid (ES); Hassane BAGHDAD KICHOU, Madrid (ES); Rita QUESADA DIEZ, Madrid (ES); Guillermo NAVARRO PATRÓN, Madrid (ES); Manuel NAVARRETE HERNÁNDEZ, Madrid (ES); Beatrice JOBST, Madrid (ES); Francesc CARANDELL VERDAGUER, Madrid (ES); Roberto Fabião SANTOS ABREU, Madrid (ES)

(21) Appl. No.: 19/478,646

(22) PCT Filed: May 26, 2023
(86) PCT No.: PCT/EP2023/064154

§ 371 Date: Oct. 24, 2025

(30) Foreign Application Priority Data
Apr. 26, 2023 (ES) P202330330

(51) Int. Cl. A61B 8/10; A61B 8/00; A61B 8/08

(52) U.S. Cl. CPC A61B 8/10; A61B 8/08; A61B 8/4483; A61B 8/5207; A61B 8/5223

(57) ABSTRACT

A system and method for non-invasive white blood cell counting in serous body fluids. The system comprises an ultrasound device that generates ultrasound images from a body or sample at a configurable spatial resolution in a scan direction, and an image processing unit that receives first ultrasound images generated by the ultrasound device at a first spatial resolution and, for each first ultrasound image, determines a region corresponding to a serous body fluid, determines a target focal point in the region, and receives at least one second ultrasound image generated by the ultrasound device focused on the target focal point at a second spatial resolution in the scan direction, higher than the first spatial resolution.

### FIELD OF THE INVENTION

The invention belongs to the field of systems and methods for estimating the concentration of white blood cells in serous body fluids.

### BACKGROUND OF THE INVENTION

Currently, the counting of white blood cells in serous body fluids (such as the cerebrospinal fluid or CSF, aqueous humour or peritoneal fluid) is carried out through manual microscopy on samples previously extracted in an invasive manner from the body [1]. An elevated amount of white blood cells (WBC) in these body fluids is an indicator of inflammatory diseases, such as meningitis (CSF), peritonitis (peritoneal fluid), uveitis (aqueous humour), chorioamnionitis (amniotic fluid) and septic arthritis (synovial fluid), among others [1].

To facilitate this procedure and to be able to perform the cell counting in a non-invasive manner, the present invention allows detecting the amount of white blood cells present in body fluids without the need to extract the serous body fluid from the human body.

### BIBLIOGRAPHY

[1] Fleming, C., Russcher, H., Lindemans, J., & de Jonge, R. (2015). Clinical relevance and contemporary methods for counting blood cells in body fluids suspected of inflammatory disease. Clinical Chemistry and Laboratory Medicine, 53(11), 1689-1706.
[2] He, K., Zhang, X., Ren, S., & Sun, J. (2016). Deep residual learning for image recognition. Proceedings of the IEEE Computer Society Conference on Computer Vision and Pattern Recognition, 2016-December, 770-778.
[3] Howard, A. G., Zhu, M., Chen, B., Kalenichenko, D., Wang, W., Weyand, T., Andreetto, M., & Adam, H. (2017). MobileNets: Efficient Convolutional Neural Networks for Mobile Vision Applications.
[4] Li, Z., Liu, F., Yang, W., Peng, S., & Zhou, J. (2022). A Survey of Convolutional Neural Networks: Analysis, Applications, and Prospects. IEEE Transactions on Neural Networks and Learning Systems, 33(12), 6999-7019.
[5] Weng, W., & Zhu, X. (2015). U-Net: Convolutional Networks for Biomedical Image Segmentation. IEEE Access, 9, 16591-16603.
[6] Ghatak, A. (2019). Initialization of Network Parameters. Deep Learning with R, 87-102.
[7] Mentis, A. F. A., Kyprianou, M. A., Xirogianni, A., Kesanopoulos, K., & Tzanakaki, G. (2016). Neutrophil-to-lymphocyte ratio in the differential diagnosis of acute bacterial meningitis. European Journal of Clinical Microbiology and Infectious Diseases, 35(3), 397-403.
[8] Tantiyavarong, P. Dialysate White Blood Cell Change after Initial Antibiotic Treatment Represented the Patterns of Response in Peritoneal Dialysis-Related Peritonitis. International Journal of Nephrology, 2016.

### BRIEF DESCRIPTION OF THE INVENTION

The present disclosure describes a methodology applied to ultrasound images recorded in different regions of the human body which make it possible to detect and count the number of white blood cells present in several serous fluids. In case of an infection, an increment of white blood cells in these fluids in the affected area can be observed, thus representing an indication of disease. The disclosure describes how these white blood cells can effectively be detected and counted in a non-invasive manner, by applying artificial intelligence methods to ultrasound images. The novel methodology herein described solves the problem of white blood cell counting in serous body fluids in a non-invasive and automatized manner.

The present disclosure refers to a system and method for non-invasive white blood cell counting in serous body fluids, such as the cerebrospinal fluid, aqueous humour, peritoneal fluid, amniotic fluid and synovial fluid.

The system comprises an ultrasound device configured to generate ultrasound images from a body or a sample of serous body fluid at a configurable spatial resolution in the direction of the scan, and an image processing unit configured to receive at least one first ultrasound image generated by the ultrasound device at a first spatial resolution in the scan direction and, from each first ultrasound image, determine a region corresponding to a serous body fluid, determine a target focal point in the region and receive at least one second ultrasound image generated by the ultrasound device at a second spatial resolution in the scan direction, higher than the first spatial resolution. The image processing unit is further configured to predict a concentration of white blood cells from the at least one second ultrasound image using an artificial neural network trained with images with an assigned ground truth concentration.

The method of non-invasive white blood cell counting in serous body fluids comprises the following steps:
Generating, by an ultrasound device, at least one first ultrasound image from a body or a sample of serous body fluid at a first spatial resolution in the scan direction.
For each first ultrasound image:
   Determining a region corresponding to a serous body fluid in the first ultrasound image.
   Determining a target focal point in the region.
   Focusing the ultrasound device on the target focal point.
   Generating, by the ultrasound device already focused on the target focal point, at least one second ultrasound image from the body or the sample at a second spatial resolution in the scan direction, higher than the first spatial resolution.
Predicting a concentration of white blood cells from the at least one second ultrasound image using an artificial neural network trained with images with an assigned ground truth concentration.

Another aspect of the present disclosure refers to a computer program product for non-invasive white blood cell counting in serous body fluids, comprising computer code instructions that, when executed by a processor, causes the processor to perform the steps of the method. The computer program product may comprise a non-transitory computer-readable storage medium having recorded thereon the computer code instructions.

### BRIEF DESCRIPTION OF THE DRAWINGS

To complete the description and in order to provide for a better understanding of the invention, a set of drawings is provided. Said drawings form an integral part of the description and illustrate an embodiment of the invention, which should not be interpreted as restricting the scope of the invention, but just as an example of how the invention can be carried out. The drawings comprise the following figures:

FIG. 1 depicts a system for non-invasive white blood cell counting in serous body fluids according to an embodiment of the present invention.

FIG. 2 depicts an ultrasound device with a single element transducer probe focused on fontanel tissue.

FIG. 3 shows two ultrasound images with different spatial resolutions acquired by an array of ultrasonic elements.

FIG. 4 represents the steps of a method of non-invasive white blood cell counting in serous body fluids according to an embodiment.

FIG. 5 is a flowchart showing, according to an embodiment, additional steps of the white blood cell counting method, including the preprocessing and quality classification of the low-resolution image.

FIGS. 6A-6D represent different preprocessing operations applied to a low-resolution image from the transfontanellar liquid space in a newborn baby.

FIGS. 7A-7D depict several examples of quality image classification of low-resolution images of cerebrospinal fluid.

FIGS. 8A-8D show some examples of quality image classification of low-resolution images of aqueous humour.

FIGS. 9A-9C depict examples of quality image classification of low-resolution images of a peritoneal dialysis bag.

FIGS. 10A-10C represents an example of manual (FIG. 10A) and automatic (FIG. 10B) segmentation of the cerebrospinal fluid, and a final segmented (binarized) image.

FIGS. 11A-11B represents an example of manual (FIG. 11A) and automatic (FIG. 11B) segmentation of the aqueous humour and the cornea.

FIG. 12 depicts the focal area of an ultrasound image acquired by a probe applied to a peritoneal dialysis bag, wherein no segmentation is needed.

FIGS. 13A-13C depict several examples of calculating the focus positioning in the cerebrospinal fluid.

FIG. 14 is an example of focus positioning in the aqueous humour.

FIG. 15 Represents the Focus Positioning in a Peritoneal Dialysis Bag.

FIG. 16 depicts the acquisition of a high-resolution image, having a higher spatial resolution than the low-resolution image, and the high-resolution image crop.

FIGS. 17A-17D depict several examples of quality image classification of high-resolution images of cerebrospinal fluid.

FIGS. 18A-18C show various examples of quality image classification of high-resolution images of aqueous humour.

FIG. 19 is a flowchart showing, according to an embodiment, additional steps of the white blood cell counting method, including the cropping, preprocessing and quality classification of the high-resolution image.

FIG. 20 shows images with different white blood cell concentrations present in the serous fluid.

FIG. 21 shows the prediction of a white blood cell concentration using a pre-trained artificial neural network.

FIG. 22 shows the calculation of white blood cell using a combination of predictions obtained for different images.

FIG. 23 shows high-resolution images with different compositions of particles mimicking the sizes of lymphocytes and neutrophils used for training an artificial neural network.

FIG. 24 shows the prediction of types of white blood cells, and their respective proportions, using a pre-trained artificial neural network.

### DETAILED DESCRIPTION OF THE INVENTION

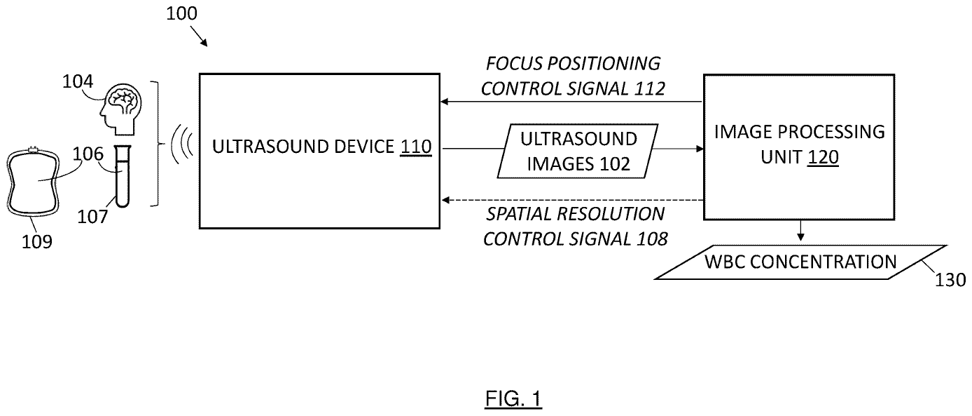

FIG. 1 illustrates the main components of a system 100 for non-invasive white blood cell counting in serous body fluids according to an embodiment of the present invention. The system 100 comprises an ultrasound device 110 and an image processing unit 120.

The ultrasound device 110 is configured to generate ultrasound images 102 obtained from a body 104 or a sample 106 of serous body fluid at a configurable spatial resolution. The body 104 may be, for instance, the head of a patient. When the ultrasound device 110 is applied to the head of a newborn baby, ultrasound images 102 of the cerebrospinal fluid located between the fontanel tissue and the cortex are obtained. Alternatively, the ultrasound device 110 can be directly applied to a sample 106 of serous body fluid previously extracted from a body 104. The sample 106 is preferably contained in a container, such as a plastic tube 107 or a plastic bag (e.g. a peritoneal dialysis bag 109). The ultrasound device 110 does not need to contact the sample 106, it may instead contact the container 107.

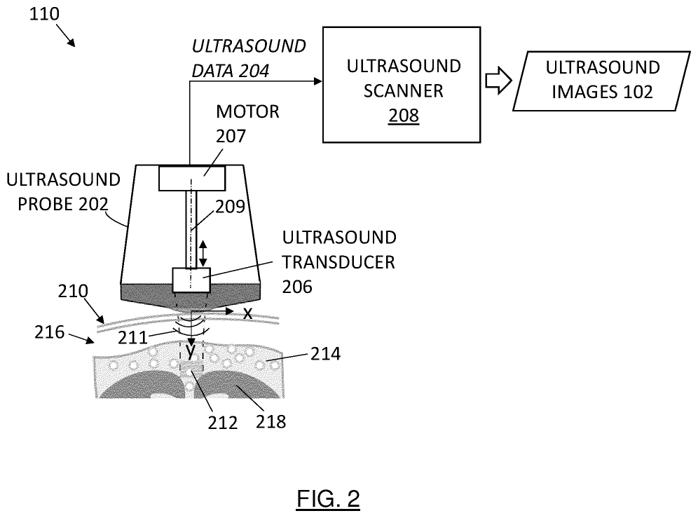

In an embodiment shown in FIG. 2, the ultrasound device 110 comprises an ultrasound probe 202 configured to acquire ultrasound data 204 from a body or sample using an ultrasound transducer 206, in acoustic contact with the body or sample, which generates and emits an ultrasonic signal through said body or sample and then receives backscattered signals. The ultrasound device 110 further comprises an ultrasound scanner 208 configured to generate the ultrasound images 102 from the ultrasound data 204, which are normally received through a wire. The ultrasound device 110 may also comprise a motor 207 (e.g. a stepper motor) configured to displace the ultrasound transducer 206 along a longitudinal axis 209 to change the focal depth (as in, for example, patent document PCT/EP2023/058245, the content of which is herein included by reference in its entirety). The image processing unit 120 may be configured to activate the motor 207, via a focus positioning control signal 112, so that the ultrasound device 110 is correctly focused, as it will be later explained.

In the example depicted in FIG. 2, the ultrasound probe 202 is applied on a surface of a baby's head 210 and acquires ultrasound data 204 for different depths (y direction, corresponding to the direction of the ultrasound wave 211 propagation) in a scan direction (x direction, perpendicular to the direction of the ultrasound wave propagation). For instance, in the ultrasound probe of PCT/EP2023/058245 a single motor 207 changes the focal depth and, once the focal depth is fixed, performs a scan in a spiral movement with high resolution steps in the axial direction (e.g. using a small thread lead), so that the focal depth is substantially kept constant across a complete rotation of the spiral trajectory. In another embodiment, the motor 207 may be in charge of adjusting the focal depth and another actuator or motor may be configured to produce a displacement (e.g. a linear displacement) in the scan direction x.

FIG. 2 represents the focal area 212 of the ultrasound transducer 206 in which the backscattered signals amplitude is maximum. The focal area 212 is located inside the superficial body fluid, in this example the cerebrospinal fluid 214 located below the fontanel tissue 216 also known as the extra-axial fluid space. When using a single ultrasonic element, such as the one depicted in FIG. 2, ultrasound data corresponding to different values in the scan direction x can be acquired by moving the ultrasound transducer 206 in said direction (the body or sample is thus scanned in the x direction), for instance by actuating a motor that moves the transducer inside the case of the ultrasound probe 202 following a predetermined trajectory (e.g. a linear trajectory perpendicular to the y direction). If the ultrasound transducer 206 is formed by an array of ultrasonic elements, electronic beam positioning can be used instead to scan the body or sample in the x direction.

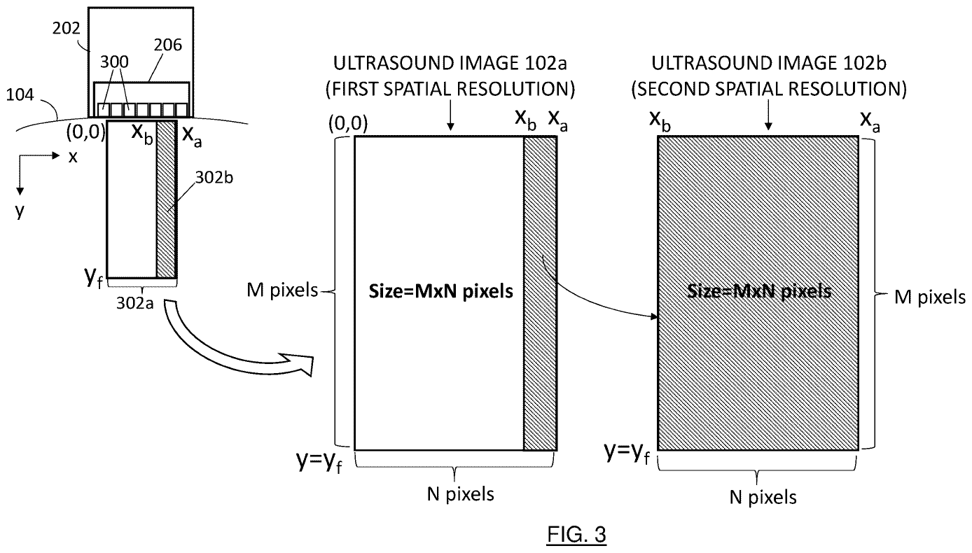

In an embodiment, the ultrasound images 102 generated by the ultrasound device 110 have the same size in pixels, M×N. However, the spatial resolution in the scan direction x of each ultrasound image 102 may vary. FIG. 3 shows two ultrasound images (102a, 102b) generated at different spatial resolutions in the scan direction x by an ultrasound device 110. Ultrasound image 102a is generated at a first spatial resolution in the scan direction x, and ultrasound image 102b is generated at a second spatial resolution in the scan direction x, higher than the first spatial resolution. The y dimension in each ultrasound image corresponds to depth in the scanned body 104 (or sample 106), whereas the x dimension corresponds to the scan direction, a direction perpendicular to the y direction on which different ultrasound measurements are sequentially acquired during a scan. When using an array of ultrasonic elements 300, as shown in the example of FIG. 3, the scan direction x is the direction on which the different ultrasonic elements 300 are arranged. When using a single ultrasonic element, as the one shown in FIG. 2, the scan direction x may be defined by the direction on which the ultrasonic transducer 206 is moved. For linear movements in the x direction, the scan direction is defined by the direction of movement; for other movements in a plane substantially perpendicular to the y direction (e.g. spiral movements in PCT/EP2023/058245), the scan direction x may be any direction perpendicular to the y direction in which there are changes in the x-value during the scan.

The ultrasound image 102a acquired at a first spatial resolution corresponds to the scanned area 302a of the body 104, which is defined between (0, xₐ) in the scan direction x and (0,y_{f}) in the y direction. The ultrasound image 102b acquired at a second spatial resolution corresponds to the scanned area 302b, which is defined between (x_{b}, zₐ) in the scan direction x and (0,y) in the y direction.

The second spatial resolution of ultrasound image 102b is higher than the first spatial resolution of ultrasound image 102a since the scanned area 302b corresponding to ultrasound image 102b is smaller than the scanned area 302a corresponding to ultrasound image 102a, and both images have the same size in pixels (M×N pixels); in particular, the scanned area 302b of the second spatial resolution is smaller in the x direction than the scanned area 302a of the first spatial resolution. In other words, the second spatial resolution is higher than the first spatial resolution since the ultrasound image 102b is formed by ultrasound data acquired in the scan direction x (i.e. a column of pixels at each x=xᵢ) in smaller steps than in the first resolution image, and therefore the scanned area 302b is shown in greater detail in the ultrasound image 102b than in the first ultrasound image 102a. In a single element transducer (FIG. 2), each column of the ultrasound image (102a, 102b) would normally correspond to a different A-line collected by the ultrasound transducer 206 at a different x-value (the spatial resolution is thus increased by acquiring A-lines at closer equidistant points).

The present invention can use any type of ultrasound device 110, provided that the spatial resolution of the ultrasound images 102 generated by the ultrasound device 110 is configurable. The spatial resolution may be configured, for instance, by changing one or more parameters controlling the electronic beam positioning when using an array of ultrasonic elements. If the ultrasound transducer 206 is a single element (FIG. 2) mechanically actuated by a motor, the spatial resolution may be configured by modifying the step of the motor in the scan direction x and by modifying the pulse rate of the transducer. If the ultrasound transducer is an array (FIG. 3), the spatial resolution may be configured by modifying the number of elements that emit an ultrasound signal and by configuring the delays of the excitation signal for each of these elements. The larger the number of elements used in the array, the higher the spatial resolution in the direction of the scan at a given depth and being the focal position in the scan direction set by the configuration of the delays of the excitation signal of each element. The image processing unit 120 may be configured to change the spatial resolution of the ultrasound images 102 generated by the ultrasound device 110 via a spatial resolution control signal 108 (an optional feature depicted in dotted lines in FIG. 1). For instance, the spatial resolution control signal 108 may control the step of a motor actuating a linear displacement of the ultrasound transducer 206 in the scan direction x, perpendicular to the longitudinal axis 209 of the ultrasound device 110, so that the spatial resolution is increased (when the motor step is smaller) or reduced (when the motor step is larger). In an array of ultrasonic elements, the spatial resolution control signal 108 may control the number of elements in the array that are excited by the driving signal, so that the spatial resolution is increased (when more elements are used) or reduced (when fewer elements are used). In addition, the delays applied to the excitation signal of each element must be configured to focalize the ultrasound beam at each of the spatial positions along the scan direction.

The image processing unit 120 can be implemented, for instance, by a processor, a GPU, a computer or a similar data processing device. The image processing unit 120 is configured to receive at least one first ultrasound image generated by the ultrasound device 110 at a first spatial resolution in the scan direction x and, for each first ultrasound image, determine a region in the first ultrasound image corresponding to a serous body fluid, determine a target focal point in the region, and receive at least one second ultrasound image generated by the ultrasound device 110 at a second spatial resolution in the scan direction, higher than the first spatial resolution, wherein the ultrasound device 110 is focused on the target focal point. The image processing unit 120 is configured to predict a concentration of white blood cells from the at least one second ultrasound image using an artificial neural network trained with images with an assigned ground truth concentration.

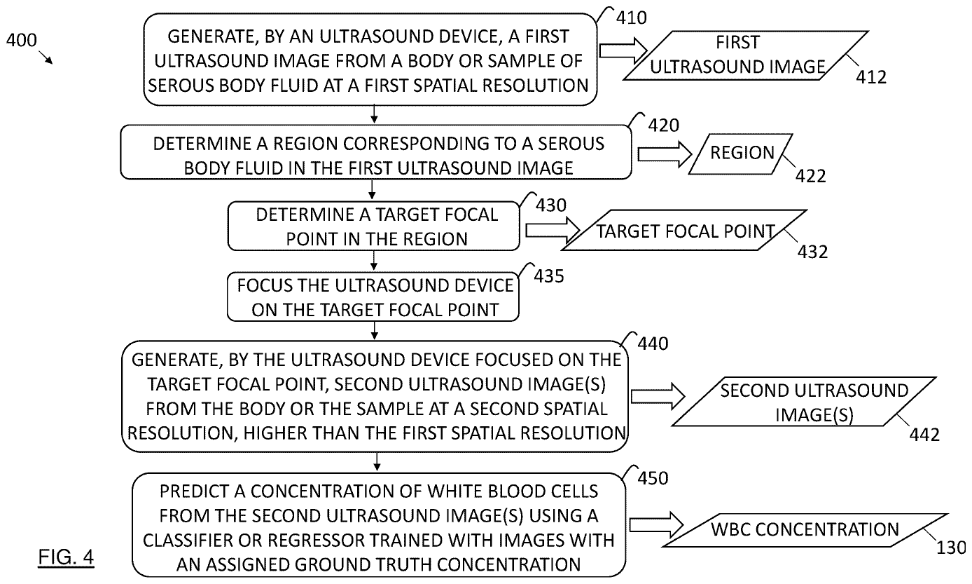

FIG. 4 is a flow diagram of a method 400 of non-invasive white blood cell counting in serous body fluids according to an embodiment. The method comprises generating 410, by an ultrasound device 110, at least one first ultrasound image 412 from a body 104 or a sample 106 of serous body fluid at a first spatial resolution. For each first ultrasound image 412, the method includes determining 420 a region 422 in the first ultrasound image corresponding to a serous body fluid, determining 430 a target focal point 432 in the region 422, focusing 435 the ultrasound device 110 on the target focal point 432, and generating 440, by the ultrasound device 110 focused on the target focal point 432, at least one second ultrasound image 442 from the body 104 or the sample 106 at a second spatial resolution, higher than the first spatial resolution. The method further comprises predicting 450 a concentration of white blood cells 130 from the at least one second ultrasound image 442 using an artificial neural network trained with images with an assigned ground truth concentration.

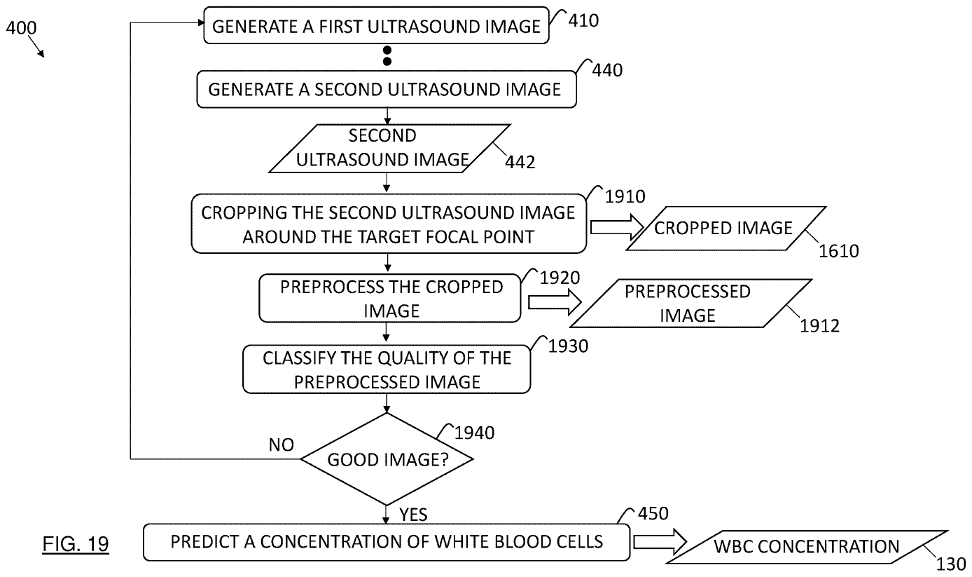

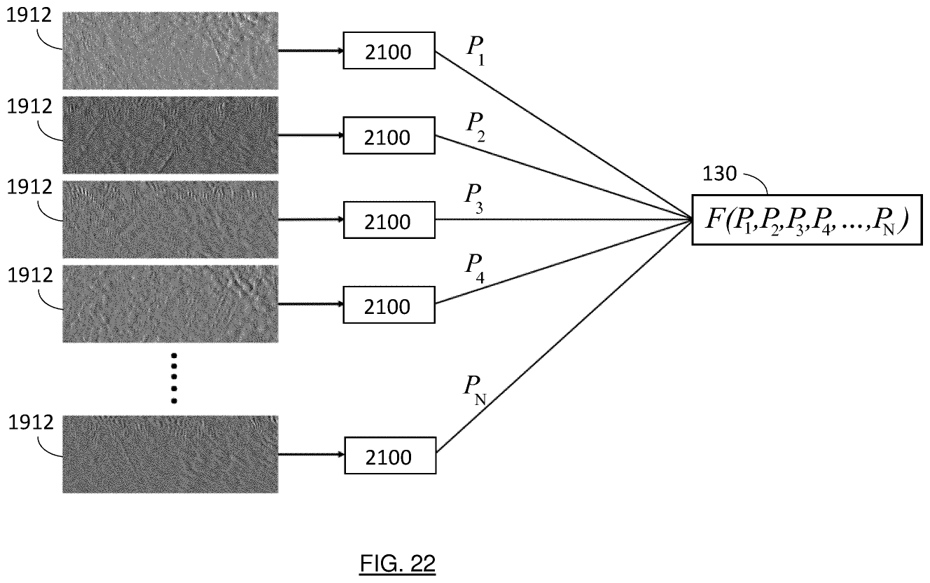

In an embodiment, a single second ultrasound image 442, obtained from a single first ultrasound image 412, may be used to predict the WBC concentration 130. However, to increase the precision in the estimated WBC concentration 130, the prediction 450 is preferably performed based on a plurality of second ultrasound images 442 (as it will be later explained in the example of FIG. 22), each second ultrasound image 442 being obtained sequentially one after the other once the ultrasound device is focused at the target focal point of a first ultrasound image 412 or, if needed due to a variety of possible problems, e.g. patient or user movement, second ultrasound images 442 may also be obtained from a different first ultrasound image 412 (the ultrasound device being therefore focused at a different target focal point) following the steps describe in the flowchart of FIG. 4 (steps 410 to 440). In other words, in the embodiment shown in FIG. 4 a single first ultrasound image 412 is used and, from that first ultrasound image 412, a target focal point is determined and one or more second ultrasound images are sequentially generated when the ultrasound device is focused on the target focal point; however, predicting 450 the WBC concentration 130 may require a predetermined number N (e.g. N=18) of second ultrasound images 442, and if said number cannot be achieved with a single first ultrasound image 412 (due, for instance, to a sudden and unexpected patient movement), further first ultrasound images 412 are generated 410 and additional corresponding second ultrasound images 442 are also generated 440 (repeating the steps 410-440) until said number is reached. Problems in the generated second ultrasound images 442 (which would be indicate, for instance, of an out of focus problem caused by a patient movement) can be detected by classifying the quality of said images, as it will be later explained in FIG. 19; if a predetermined number of bad images are sequentially detected (e.g. 5 consecutive bad images), the process can be restarted, generating 410 another first ultrasound image 412 to obtain another target focal point 432 for generating 440 additional second ultrasound images 442.

The method 400 thus employs a two-step process. Firstly, a low-resolution (LR) image (i.e. first ultrasound image 412) is acquired to locate and segment a fluid region 422 corresponding to the serous body fluid and determine a target focal point 432 within said fluid region 422. Secondly, the focus of the ultrasound transducer 206, formed by a single ultrasonic element or an array of ultrasonic elements, is positioned at the target focal point 432 within said fluid region 422 and one or more ultrasound images of the selected region of interest within the fluid is acquired at an increased spatial resolution, high-resolution (HR) image(s) (i.e. second ultrasound image(s) 442). Then, the concentration or number of white blood cells in the high-resolution images present in the fluid are estimated using artificial intelligence models, such as deep learning. The terms “low-resolution images” and “high-resolution images” used in the present disclosure refer to images which, although they may have the same size in pixels (same graphical resolution), they have different spatial resolution in the scan direction, high-resolution images corresponding to a higher spatial resolution than low-resolution images (meaning that the high-resolution image corresponds to an area scanned by the ultrasound device smaller than the area scanned for the low-resolution image but formed by a higher number of equidistant acquisitions along the scan direction x).

This method can be completely automatized. Throughout the whole process, different types of artificial intelligence models are applied to automatically associate images or parts of images to different classes or groups or to automatically segment specific structures in the images. In an embodiment, all these models have an overall architecture in common, deep convolutional network models, which pass the images through several different layers to automatically learn different features and connections between certain aspects of the images, thus being able to automatically associate the images to a specific group or class. Examples of deep learning architectures that can be applied in the various models are the Resnet architecture [2], the MobileNet architecture [3], or a general convolutional neural network architecture [4], but the method is not limited to these architectures.

The ultrasound images 102 are preferably acquired in B-mode scan configuration. B-mode images can be produced by 1-D, 2-D, or 3D arrays of ultrasonic elements that use electronic beam positioning to conform the image pixel by pixel, or by single element transducers that are mechanically actuated to scan a linear or any other trajectory at a substantially constant focal depth. In particular, the scan mode of a mechanically actuated transducer scanning a fan-shape image is known as C-scan. A mechanically actuated transducer collects one or more A-lines at each spatial location to produce at the end of the scan, a B-mode image. An array also collects B-mode images by moving the position of the focus to sample a 1-D, 2-D, or 3-D region.

Like b-mode scans, m-mode (motion mode) scans produce 2D, 3D or 4D images but in this case the probe enclosing all the transducer elements is fixed and always firing at the same location. This scanning mode allows high acquisition speed and, therefore, detection of motion in the media. In the absence of motion, the scan is simply called an A-scan and is often referred to the acquisition of A-lines at a fixed position.

In an embodiment, the ultrasound device 110 comprises a pen-like single focused-element transducer probe 202 whose tip has a circular shape and the trajectory of the transducer inside the probe is substantially parallel to the outer plane of the tip all along the scan trajectory (i.e. the transducer can scan an area at a substantially invariable focal depth, as described for instance in patent document PCT/EP2023/058245). At each discrete position of the scan, the transducer transmits one or more signal pulses and produces one A-line from all the echoes received at the excited location. At the end of the scanned trajectory (e.g. a linear trajectory), the scan starts again but in the opposite direction. To adjust the focal position to inspect a target tissue, the transducer can be displaced axially by means of a motor or actuator.

Although the raw A-line contains positive and negative voltage amplitudes corresponding to the echoes produced from the different structures present along the A-line position, A-lines are commonly bandpass-filtered and Hilbert-transformed to work only with the envelope of the signal. As a result, pixel intensity values correspond to absolute amplitudes of echoes from structures encountered by the transmitted signal along the A-line location. B-mode images are normally represented in gray scale.

Unlike diagnostic imaging where the exact location of a structure of interest is not known and the user must navigate the probe to locate such region of interest, within the framework of the present disclosure the user knows beforehand where the fluid to be inspected is located. Generally, the user places the probe on the tissue, keeps it still and activates the probe to start the measurement of cells in the fluid right below. Only if the probe has been mistakenly placed or if there is too much hair gel or water needs to be applied, should the probe be lifted and rested again on a slightly different and close location to restart the procedure. All in all, the probe is not meant for navigation but to reach much sensitivity as a point of measurement—white blood cell counter—device.

Since the ultrasound transducer 206 is a focused transducer, the acoustic pressure is mostly concentrated in the focal point at a fixed focal distance. In an embodiment, the transducers used have focal distances centered at about 14, 17 and 19 mm and their focal depth (i.e. depth of the focal area or focal region) is about 2 mm for each of them, Although the transducers may have different focal distances and focal depths.

Unlike array systems that display images of a fixed region to the user and where the user may adjust the focal distance dynamically on the screen without changing the field of view of the image, the system 100 is not intended to show images to the user and preferably represents the focal area at the middle of the image. Therefore, when the ultrasound transducer 206 is moved towards the body 104 or sample 106 the image is scrolled up, and if the ultrasound transducer 206 is moved away from the body 104 or sample 106, the image is scrolled down; as a result, the theoretical focal region is always kept centered in the middle of the image.

Although the focal distance of the image may vary from the actual focal distance because of the beam distortions implied by the tissue crossed by the travelling signal, by representing the focal area in the middle of the image the information around the focal point is highlighted and can be more easily analysed and, in addition, images of smaller height can be generated.

At the focal point is where the beam reaches a higher acoustic pressure amplitude (and intensity) and, therefore, is in this region where the transducer is most sensitive to small structures, e.g. cells. At the driving signal configurations used, the acoustic pressure is enough so as to induce a displacement in the cell away from the transducer, that is, the acoustic signal “pushes” the cell. This phenomenon is called the acoustic radiation force.

Interestingly, by moving the transducer at very small steps in the scan direction x (high resolution images) from one A-line location to its adjacent position, the acoustic beams between A-lines overlap and the acoustic “push” to the cell can be repeated several times until the cell is driven out of the focal region, i.e. about 1 mm below the focal distance. Likewise, the cell is induced into the focal region by the acoustic beam and accelerates as it is being dragged towards the actual focal point. When it leaves the focal center, the cell decelerates and the pushing pressure is also lower as it is located further from the focal center. This induction-acceleration-deceleration-ejection that happens in a set of consecutive A-lines to the cell across the focal region leaves a characteristic pattern that proves essential to discriminate cells from noise and, consequently, to reach a clinically acceptable level of sensitivity to abnormal serous fluids. When using arrays, the same effect can be accomplished by electronically focusing at close locations along the scan direction x. The central frequency of the ultrasound transducer is preferably equal or higher than 15 MHz, this frequency would be enough to detect cells at an individual level. In the dialysis bag use case, frequencies up to 40 MHz may be used, or even more. In the CSF and aqueous humour use cases the selected central frequency is around 20 MHz, but this frequency could vary (e.g. up to 25 Mhz).

The different steps of the method 400 will be hereinafter described in detail using several examples and use cases. In particular, three different use cases of white blood cell (WBC) counting are considered (although the method 400 and system 100 can be applied to other use cases in which WBC counting in serous body fluids is required):
WBC counting in the cerebrospinal fluid (CSF), wherein the ultrasound device 110 is applied to the head of a patient (e.g. baby's head 210).
WBC counting in the aqueous humour, wherein the ultrasound device 110 is applied to an eye of a patient.

WBC counting in the peritoneal fluid, wherein the ultrasound device 110 is applied to a sample 106, in particular to a peritoneal dialysis bag.

Before the target fluid is identified, a low-resolution ultrasound image (e.g. with a size 1600×730 pixels acquired at a sampling rate of 100 MHz), which correspond to the first ultrasound image 412 at a first spatial resolution, can be automatically classified according to its quality to dismiss poor quality images. The classification in good or bad quality images first comprises common pre-processing operations of the ultrasound images. In an embodiment, the pre-processing operations include the following steps: the signal is filtered by a Butterworth bandpass filter (e.g. of 6^{th} order between 15 and 25 MHz); then, the Hilbert transform is applied to obtain the signal envelopes which are then used for further processing; the dynamic range of all images is adjusted between a minimum and a maximum value, applying a min-max normalization. Finally, the image size may be reduced (e.g. to 256×128 pixels) to reduce the computation time. After the preprocessing steps, a deep learning model based on a convolutional neural network architecture is trained to automatically classify the preprocessed image into one of the pre-established classes.

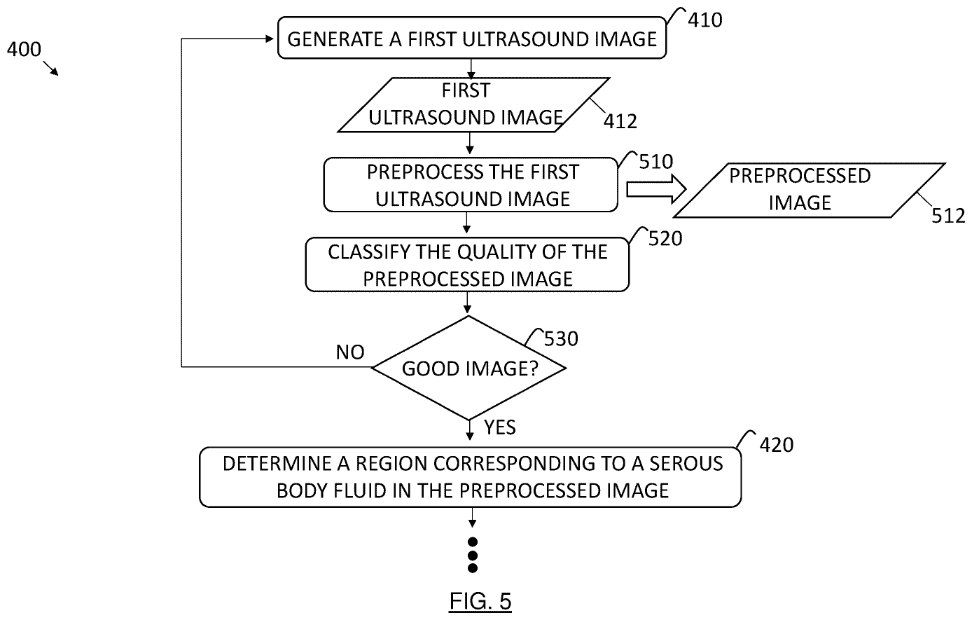

FIG. 5 is a flowchart of the method 400 according to another embodiment, showing optional steps leading to a quality classification process carried out by the image processing unit 120. According to this embodiment, the method 400 further comprises preprocessing 510, by the image processing unit 120, the first ultrasound image 412 to obtain a preprocessed image 512 prior to determining the region corresponding to a serous body fluid in said image. In an embodiment, the preprocessing 510 includes applying a bandpass filter, a Hilbert transform, a min-max normalization and, optionally, an image size reduction.

The method 400 may also comprise classifying 520, by the image processing unit 120, the quality of the preprocessed image 512 into a class from a set of predetermined classes, using an artificial neural network trained with manually labelled preprocessed images, and proceed to determining 420 the region corresponding to a serous body fluid only when the preprocessed image 512 is classified into a predetermined class (or classes). In the example, it is checked 530 whether the preprocessed image 512 is classified as “Good image” (i.e. good quality image); in that case the process continues with the next step 420, otherwise the quality classification process starts again by generating 410 and analysing a new fist ultrasound image 412 (the method may include sending a warning to the user, e.g. a “Bad image” message, to reposition the ultrasound probe).

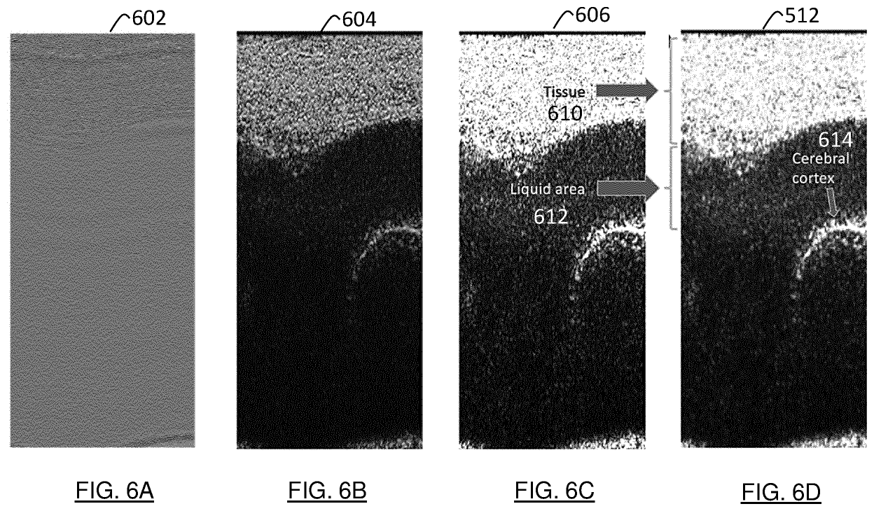

FIGS. 6A-6D show different steps of the preprocessing operations applied to an exemplary first ultrasound image 412 acquired at a low resolution from the transfontanellar liquid space in a newborn baby. FIG. 6A depicts the raw image 602 obtained prior to the preprocessing, which corresponds to the first ultrasound image 412 generated by the ultrasound device 110. FIG. 6B shows the previous image after applying the bandpass filter and the Hilbert transform (image 604). FIG. 6C represents the image of FIG. 6B once the dynamic range is adjusted using the min-max normalization (image 606). The min-max normalization serves to saturate the image to see better the anatomical structures and the patterns of the moving cells. The minimum and maximum values are preferably heuristically chosen and are common to all images (e.g. the minimum is set to 1000 and the maximum to a value between 18000 and 23000). FIG. 6D shows the preprocessed image 512 used for classification after the image size reduction process. The preprocessing operations may include all of these operations or a combination thereof (e.g. the image size reduction may be optional). The white region in the upper part of the image are the different tissue 610 types (epidermis, dermis and meninge), followed by the liquid area 612 below. Tissues 610 can hardly be visually differentiated among them after the intensities are normalized. The fluid space, or liquid area 612, is delimited below by the cerebral cortex 614 (white curved line).

Once the preprocessing operations are finished, the preprocessed image 512 is classified, using a pre-trained artificial neural network (e.g. a convolutional neural network), according to its quality into one of a set of pre-established classes, the set of pre-established classes including at least a class corresponding to good quality images.

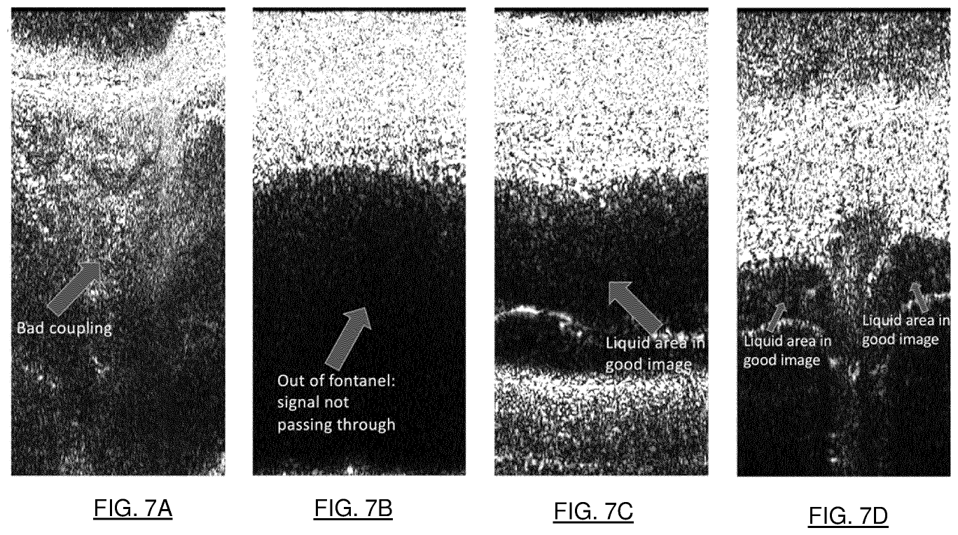

FIGS. 7A-7D depict different examples of quality image classification for the use case of cerebrospinal fluid. Ultrasound images of the cerebrospinal fluid 214 located between the fontanel tissue 216 and the cerebral cortex 218 are recorded first in an exploratory, low-resolution mode (first ultrasound images 412 at a first spatial resolution). These low-resolution images inform of the optimal area where high resolution images (second ultrasound images 442 at a second spatial resolution) should be collected to resolve and count the white blood cells present in the liquid. An elevated number of white blood cells present in the cerebrospinal fluid can be used, for instance, as an indicator of meningitis.

In an embodiment, for the low-resolution images three different types of image classes are established: “Bad coupling”, “Out-of-fontanel” and “Good image”. With this classification model it is automatically decided whether an image is of bad quality (bad coupling, out of fontanel) or of good quality in order to continue with the procedure of obtaining an estimation of the white blood cells present in the fluid. In case of a bad quality image, the user is alerted (e.g. via an acoustic alarm, a visual alarm, a notification, etc.) to improve the acoustic coupling with tissue and make sure that the probe is not tilted and is correctly positioned onto the cranium-free fontanel region.

Unlike the “Bad coupling” class that can be used to remind the user not to tilt the probe and make sure the acoustic coupling with tissue is good, the “Out-of-fontanel” class allows to inform the user that the probe must be repositioned to avoid the forming cranium under the skin.

Particularly, the “Bad coupling” class represents images where the coupling between the device and the skin is not optimal, producing noisy ultrasound images that cannot be used (see example of FIG. 7A). The “Out-of-fontanel” class represents images where the device is not placed or is only partially placed onto fontanel region with the cranium preventing, thus, the signal from reaching the fluid and the cortex (see example of FIG. 7B). The “Good image” class represents valid images, where there is fluid area at a good signal quality that allows for switching to a higher resolution mode for the cell counting step, see examples of FIG. 7C (an off-sagittal sinus location) and FIG. 7D (where the ultrasound device is positioned above the sagittal sinus, a major component of the superficial cerebral venous system; the lateral liquid space next to the sagittal sinus is a good area for acquiring high-resolution images).

After labelling the training data manually with these three classes (although different types and number of classes may be employed in the training, for instance “Good image” and “Bad image”), a deep learning model is trained, which is able to resolve the task to automatically classify images into the aforementioned classes. In an embodiment, the deep learning model architecture is based on a modified Resnet50 network architecture which adds one more convolutional layer before the output layer. In this case the network does not contain pre-trained weights. The model is optimized according to the lowest value of the loss function obtained after optimization on the training set. The strategy to train the model is based on a k-fold cross validation technique where the model is always trained on 80% of the data (80% from all patient images collected in the dataset) and validated on the remaining 20%. In the next round of training, another combination of patients' data accounting for 80% of the dataset is used to train the model and the corresponding other 20% of data is used to validate and so on, until having had all patients once in the validation data set. This way the generalization ability of the model can be checked. The input of the model is a single image and the output is a probability for belonging to each of the predetermined classes. The predicted class is chosen based on the highest probability value.

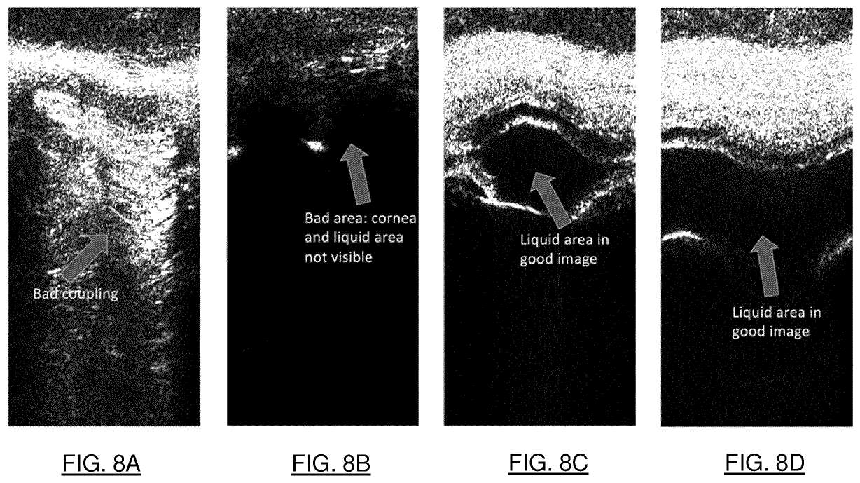

FIGS. 8A-8D depict other examples of quality image classification for the use case of aqueous humour. Ultrasound images of the liquid space in the anterior chamber between the cornea and the crystalline lens are acquired. The goal is to estimate the amount of white blood cells present in the liquid, the aqueous humour, where an elevated number of white blood cells present can be used, for instance, as an indicator of uveitis, an inflammation of the middle layer of the eye, also called uvea. The same procedure as in the CSF case is applied, in this case also using only a two-class model, “Good image” and “Bad image”. “Bad image” class groups poor quality images due to bad coupling, excessive tilting of the probe or an incorrect position of the probe, far from the target area. Examples of bad quality images are shown in FIG. 8A (bad coupling with the ultrasound device) and FIG. 8B (wrong area, the liquid space in the anterior chamber is not visible). “Good image” class examples are shown in which the liquid space in the anterior chamber can be seen from a lateral plane of the eye (FIG. 8C) or from a midline plane of the eye (FIG. 8D). The images that are not good enough for scanning in high resolution mode can be manually labelled as “Bad image” and can be used to train the classification model to distinguish between good and bad quality.

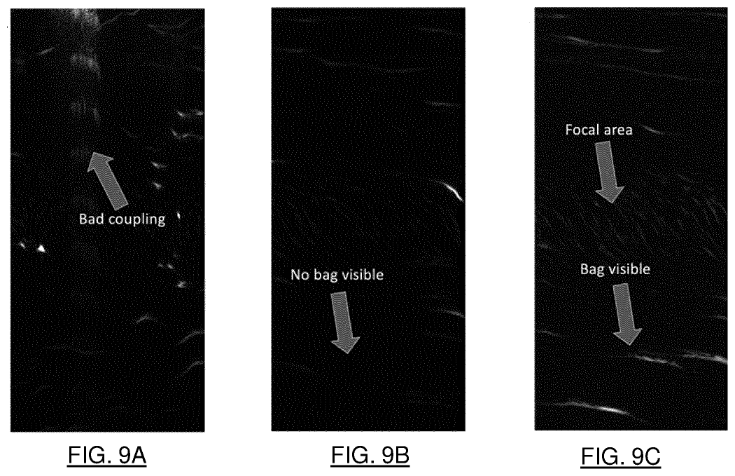

FIGS. 9A-9C show other examples of quality image classification for the use case of peritoneal fluid contained in a peritoneal dialysis bag 109. Ultrasound images are non-invasively acquired directly from the peritoneal dialysis bag 109, with the goal of estimating the amount of white blood cells present in the peritoneal fluid, wherein an elevated number of white blood cells in the liquid can be an indicator of an infection. The same procedure as in the CSF and aqueous humour is applied to an image quality classifier trained to distinguish between Good image and Bad image quality. In this case bad image quality can mean a bad coupling (FIG. 9A) or that there is no reference (border of the peritoneal dialysis bag 109) visible in the acquired image (FIG. 9B, the border of the bag should be visible in the area where the arrow is pointing at). A good image quality example is shown in FIG. 9C, where high resolution images can be acquired. A deep learning model (e.g. a convolutional neural network) is trained using previously manually labelled low-resolution images.

Back to FIG. 4, the step of determining 420 a region corresponding to a serous body fluid in the first ultrasound image 412 (or in the preprocessed image 512, when the ultrasound image 412 is preprocessed 510) may include segmenting a region in the image corresponding to a liquid area. The segmentation is performed by an artificial neural network previously trained with images including manually segmented regions. In the method 400 of FIG. 5, a deep learning model automatically segments an area of interest in the images classified as good image quality, the deep learning model being trained with images in which a specific area of interest is manually segmented.

In the embodiment, the applied deep learning model used for segmentation of selected image regions is based on a U-Net architecture [5], with an encoder and a decoder branch with a network weights initialization, i.e. Xavier's initialization method, scheme for each layer [6]. The models are trained following a k-fold cross validation approach as described in the section before. With the training of this model, the fluid area can be automatically segmented.

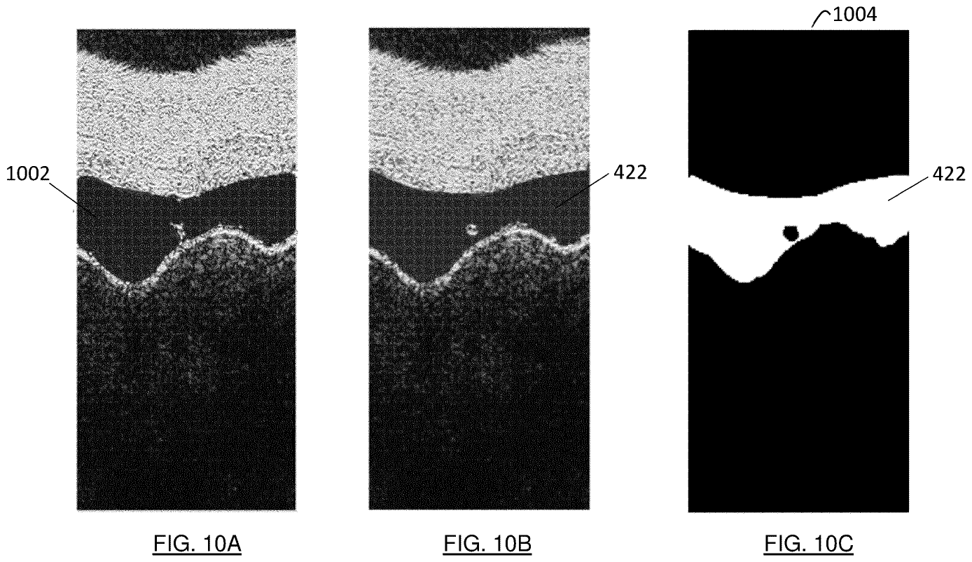

FIG. 10A shows an example of the manually segmented liquid area 1002 in case of the tranfontanellar space for the meningitis screening application used for the training of the artificial neural network. FIG. 10B represents the region 422 automatically segmented by the trained artificial neural network. The output of the trained artificial neural network (segmentation model) applied to an image (e.g. of 256×128 pixels) is an image with the same size consisting of values between 0 and 255 for each pixel. The higher the pixel intensity value the higher the probability of that pixel to belong to the segmented area. Arbitrarily, a threshold (e.g. of 50) is set upon which the image is then binarized, meaning that for all values below the threshold (e.g <50) the value is set to 0 (black pixels) and for all values equal or above the threshold (e.g. >50) the value is set to 1 (white pixels). This way a final segmented image 1004 with the segmented region 422 or area of interest (white area) is obtained, as depicted in FIG. 10C.

In the CSF use case, the artificial neural network is trained to automatically segment a region 422 corresponding to the cerebrospinal fluid area for finding within this region 422 an optimal zone to acquire images in high resolution mode for the cell counting task. Again, images are manually segmented to train a deep learning model based on the above-described U-Net architecture to automatically generate segmented images.

In the aqueous humour use case, the artificial neural network is trained to automatically segment a region 422 corresponding to the fluid in the anterior chamber between the cornea and the crystalline lens. The process is implemented in the same way as with the CSF use case.

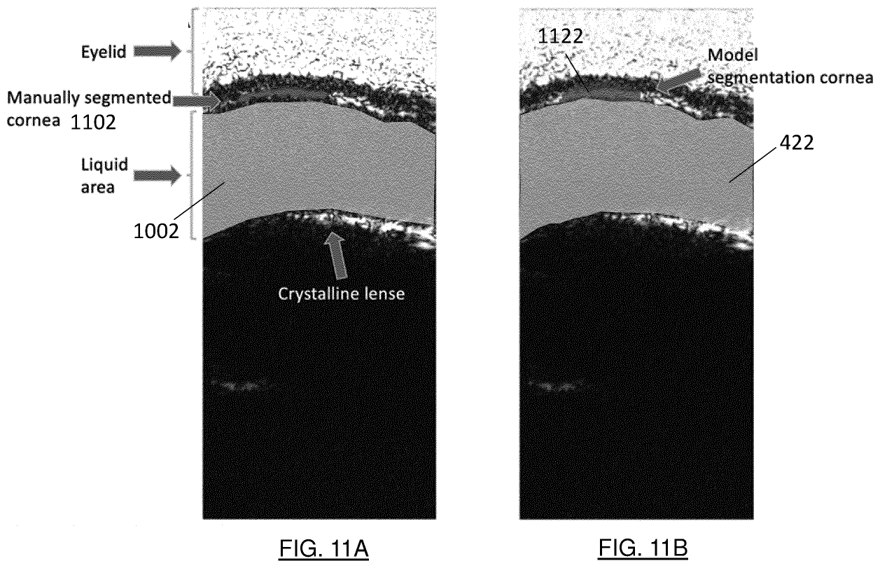

Remarkably, a neat visualization of the cornea informs about the good signal quality in the fluid region 422 below and, as a result, defines the best region to switch to high resolution for cells visualisation. This “cornea check” criteria to pick the best region would be equivalent in the CSF use case by studying the parietal arachnoid layer, which is the interface layer between the fontanel tissue and the CSF. In a more generalized way, the interface layer between the tissue and the fluid provides a neat reflective signal that is, therefore, well defined, continuous, as free from clutter noise as possible and of a high amplitude when compared to signals coming from the fluid. These features ensure both that the coupling and the tilt of the ultrasound probe are the appropriate for the signal to reach the target area in best possible conditions. Therefore, in the aqueous humour use case a first region 422 corresponding to the liquid area is segmented and, in addition, a second region 1122 corresponding to the cornea is also segmented (FIG. 11B).

FIG. 11A shows an example of the manually segmented cornea 1102 (the part of the image where the cornea is clearly visible and continuous) and the manually segmented liquid area 1002 used for the training of the artificial neural network. The procedure for the automatic segmentation of the cornea follows the same steps as the liquid segmentation.

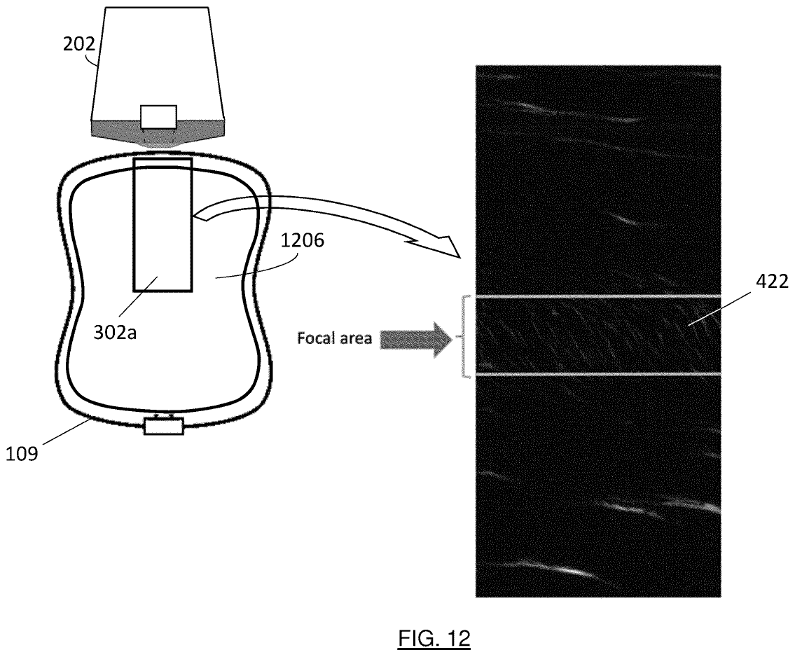

In the use case of the peritoneal dialysis bags 109, no segmentation is applied, since the bag content is all fluid with no other structures and, as a result, the focal point always falls within the peritoneal fluid 1206 (FIG. 12) and if a fluid model was applied the whole image would be masked. In an embodiment, the region 422 determined in step 420 corresponds to the focal area of the ultrasound probe 202. In the example of FIG. 12 the focus is positioned at the height of half of the image size (in y-direction), which represents the area of interest.

The different applications where the device is directly applied on a human body described for segmentation of regions 422 or areas of interest corresponding to a liquid area all have in common the same type of procedure, where a specific region of interest in the low-resolution images is automatically segmented after training a deep learning algorithm on previously manually-segmented images. The same procedure could be applied to any kind of structure in the images (in other applications other parts of tissue could be of interest, but the methodology remains the same).

The information extracted from this segmentation technique is used in a next step to automatically find the optimal region where to switch to high resolution mode. The information can also be used to measure relevant parameters considered in the subsequent models to optimize the accuracy of the final cell count model: tissue thickness to estimate attenuation, high intensities in a specific region of the cornea as a best region for measuring beneath, artifacts (vessels and other abnormalities) detection, etc.

Remarkably, these algorithms have the potential to enable the measurement of other relevant biomarkers of infection, such as the thickening of the arachnoid layers (in the case of meningitis) that embrace the cerebrospinal fluid as an inflammatory response to the passage of white blood cells from the bloodstream to the cerebrospinal fluid.

Once the region 422 corresponding to the fluid space is identified, the acoustic beam focus is placed within this region to ensure that the maximum pressure is going to hit a cell suspended in the area and, therefore, a portion of such pressure wave is going to be reflected back and sensed by the transducer.

Ideally, the focal region should be located 2 mm below tissue to minimize the amount of clutter noise (tissue reverberations) that can potentially confound counting models. For ultrasound transducers 206 generally used for imaging and formed by an array of ultrasonic elements 300 (FIG. 3), the focus can be adjusted electronically; the focusing of the ultrasound device on the target focal point may be performed either by the image processing unit 120 via the focus positioning control signal 112 or by a user inputting the focal point on a user interface of the ultrasound device 110. In the case of a fixed-focus single-element transducer probe 202 (FIG. 2), the focus can be moved axially by a mechanical displacement of the ultrasound transducer 206 automatically by a motor controlled by the image processing unit 120 through the focus positioning control signal 112. The axial displacement amount to be applied to the transducer is calculated based on a target focal point 432 within the segmentation mask of the liquid area (i.e. region 422). The image processing unit computes the target focal point 432 within the region 422 on a case by case basis.

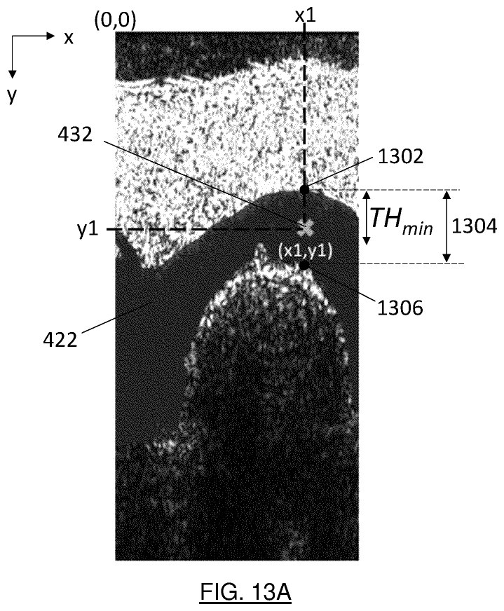

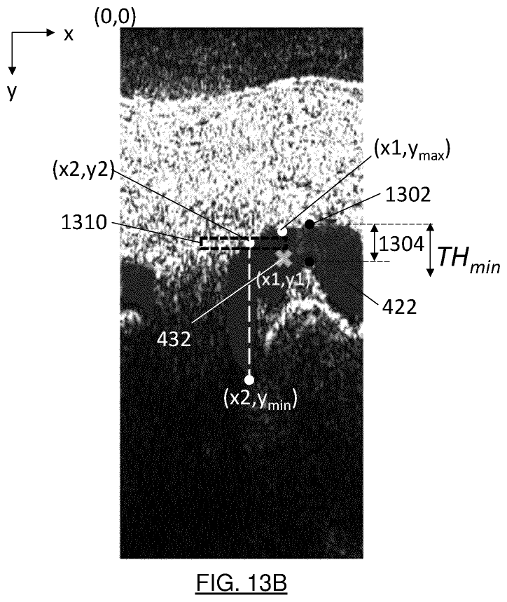

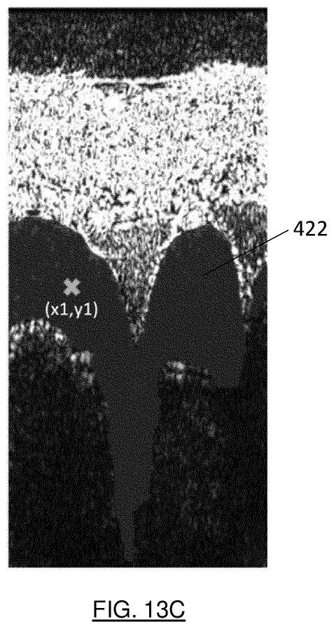

The method 400 comprises focusing 435 the ultrasound device 110 on the target focal point 432. The image processing unit 120 controls the focusing of the ultrasound device 110 via a focus positioning control signal 112, so that the ultrasound device 110 is focused on the target focal point 432. With the transducer focus centered at the target focal point 432, the high-resolution scan is initiated in the x direction by acquiring A-lines that are equidistant and close between them across a small area. If using arrays, the focus is moved at the same equidistant positions by configuring a set of delays of the excitation signal for each of the elements of the array that are actuated and for every position along the scan direction. In the CSF use case, the liquid area (region 422) can adopt different shapes, as shown in the examples of FIGS. 10B, 13A, 13B and 13C. The focus should optimally be put in a position with enough liquid space (in y-direction), to guarantee being able to detect the cells—if present—moving within the liquid. A minimum desired liquid space is 2 mm although the models have proved to work at thinnest spaces.

In case of the liquid space having the shape as in FIG. 10B (an off-sagittal sinus location), basically any point of the segmented area in x-direction can be used. On the other hand, in case of the liquid space having the shape as in FIG. 13C with the sagittal sinus present in the image (the V-shaped structure), it is best to avoid placing the focus below the sagittal sinus, as this vessel attenuates largely the signal and would limit the signal arriving to the cells in the liquid.

In an embodiment shown in FIG. 13A, the strategy for calculating the target focal point starts by running through all image pixel columns in x-direction and computing the highest point 1302 (in y-direction) of the whole segmented liquid area (region 422). At the highest point in y-direction, it is checked that this point is not an outlier produced by an erroneous segmentation but an actual part of the main liquid mask. To do so, below the highest point 1302 there needs to be a thickness 1304 of the segmented area equal or larger than a minimum thickness THₘᵢₙ (e.g. a thickness 1304 equal or larger than 20 pixels). Then, the difference in y-direction between the highest point 1302 and the lowest point 1306 is computed to obtain the thickness 1304 in the vertical direction of the liquid area at the established point on the x-axis (x1). The target focal point 432 is then defined by x1 and y1, the y-value y1 representing the middle point of the fluid thickness at that x1 position, computed as the y-value of the highest point 1302 of the segmented area at x1+½ thickness 1304.

If, on the contrary, the highest point 1302 does not fulfil the thickness check described above (i.e. the thickness 1304 is lower than the minimum thickness THₘᵢₙ), as shown in the example of FIG. 13B, the x-value of the lowest value ymin of the region 422 (segmented area) in y-direction is computed (x2). The highest y-value of the region 422 at x2 is calculated (y2). The target focal point 432 should be located away from the sagittal sinus. The focus positioning algorithm looks for the highest segmented point in a window 1310 of a predetermined size similar to that of the sagittal sinus width (e.g. a window of 160 pixels, 80 pixels to the left in the x-direction and 80 pixels to the right in the x-direction from (x2,y2)). The highest point in the image pixel column located at 80 pixels to the left from x2 is calculated. If the y-value of this highest point at x2−80 is larger than y2, the focus positioning algorithm overwrites y2 with the y-value of this highest point. By finding higher y-values at each step the focus positioning algorithm is “escalating” the sides of the sagittal sinus. The focus positioning algorithm now sweeps to the right of the window pixel by pixel repeating the described process, reaching the point at 80 pixels to the right from x2. If the y-value of the highest point during the sweep is larger than y2, the focus positioning algorithm overwrites y2 with this new y-value. The x-value (x1) at the highest y-value (in the example, yₘₐₓ) corresponds to the x-value of the target focal point 432. The target focal point 432 (x1,y1) is then calculated as in the first strategy, the y-value being selected as the mid-point at x1 and it is computed as the y-value at the highest point (yₘₐₓ) of the segmented area at x1+½ thickness.

Therefore, the target focal point 432 in the transfontanellar liquid space in the example of FIG. 13A is computed based on where the highest point in the region 422 is located (in y-direction), whereas in the example of FIG. 13B the focus is computed as the highest point (in y-direction) of the region 422 in an area of +/−80 pixels around the lowest segmented area (in y-direction). With this strategy, the target focal point 432 is located in an area with sufficient liquid space and is not right below the sagittal sinus. An example of the focus positioning with the sagittal sinus present is shown in FIG. 13C, using the described focus positioning algorithm.

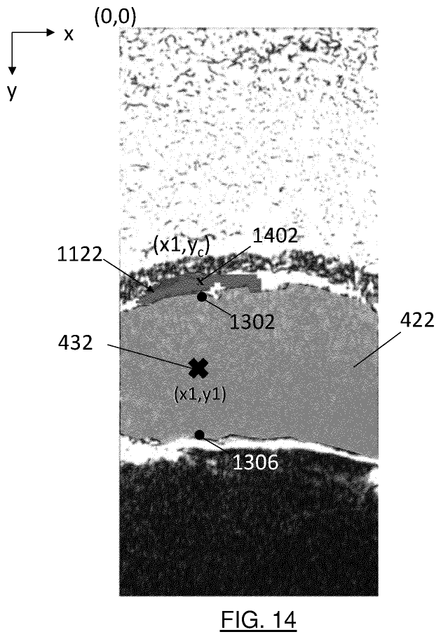

In the use case of the liquid in the anterior chamber between the cornea and the crystalline lens (focus positioning in the aqueous humour, FIG. 14), the target focal point is calculated based on the segmentation of the liquid area (region 422 and on the segmentation of the cornea (second region 1122). The x-value (x1) for the target focal point 432 is calculated as the middle point 1402 (x1,y_{c}) in the cornea segmentation area, second region 1122. At this x-value (x1) the highest point 1302 and lowest point 1306 of the liquid segmentation (region 422) in y-direction is calculated and the y-value (y1) of the target focal point 432 is then set as the highest point 1302 of segmented liquid area+½ thickness. Therefore, the focus is put in the x-direction at the central point of the cornea segmentation and in the y-direction in the middle of the liquid of the anterior chamber calculated as the highest point of segmented area in y-direction+½ thickness (the thickness being defined as the y-value of the highest point 1302 minus the y-value of the lowest point 1306 of segmented area in y-direction).

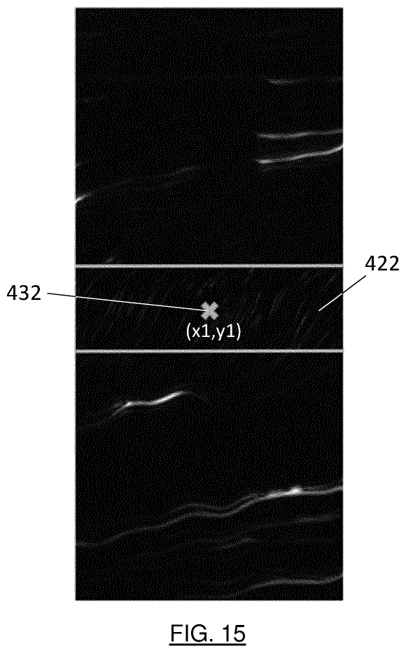

In the use case of peritoneal fluid contained in a peritoneal dialysis bag 109, the target focal point 432 is set as the central point of the region 422, which corresponds to the focal area of the ultrasound probe 202. Since the focal area is centered in the image, the target focal point 432 also corresponds to the central point of the low-resolution image in x and y direction, as shown in FIG. 15.

In the described use cases the positioning of the focus within the area of the liquid (region 422) is defined based on the liquid segmentation (e.g. shape and size) and, optionally, other anatomical structures or tissues (e.g. second region 1122 corresponding to the segmented cornea), depending on the application.

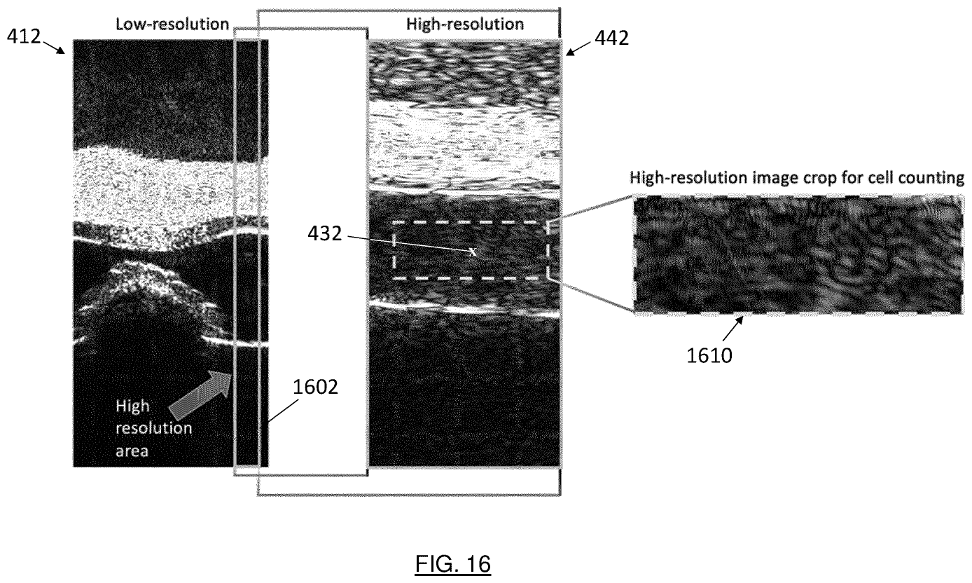

Once the optimal area is identified for acquiring high-resolution images, the focus of the ultrasound device 110 is moved to the specified x-y coordinates (x1,y1) defining the target focal point 432 computed as described above. Then, images are recorded at a second spatial resolution with the same size as low-resolution images (e.g. 1600×730 pixels), but the second spatial resolution is higher than the first spatial resolution (e.g. 8 times higher in the x-direction in the example shown in FIG. 16) compared to low-resolution images. In case of arrays, an increase of spatial resolution in x direction is accomplished by using a larger number of neighbour elements that are excited at the right pulse delay configuration to allow focusing at closer locations in the x-direction.

The target focal point 432 defines the start of the high-resolution image, since the ultrasound device is focused 435 (FIG. 2) on the target focal point prior to generating the second ultrasound image 442. In the embodiments of FIGS. 13, 14 and 15, the calculated target focal point 432 may be the focusing point; alternatively, the final target focal point (on which the ultrasound device is focused) may be the calculated target focal point displaced to the left or right, so that the high-resolution image is substantially centered on the calculated point after the scanning process.

In an embodiment, the image processing unit is configured to crop the second ultrasound image 442 around the target focal point 432 (in the center of the image) and predict the concentration of white blood cells 130 in the cropped image 1610. The cropping is however optional, since the artificial neural network used for the cell counting may be trained using whole high-resolution images (second ultrasound images 442) instead of cropped images 1610. If cropping is applied, for the next steps only the focal area of the images is taken into consideration, which is delimited by the cropped section (e.g. in FIG. 16 the cropped image 1610 is delimited in y-direction by the rows comprised between 100 pixels above and 100 pixels below the focal line, and in x-direction by the columns located 100 pixels from the left border and 74 pixels to the right border, resulting in images of the size 200×556 pixels).

While the cropping of the images in the y-direction aims at including only signals received from structures located in the theoretical focal region, the cropping in the x-axis responds to the removal of random friction artifacts that may occur inside the ultrasound probe due to bouncing of the transducer when reaching the ends of the trajectory. These friction artifacts do not occur on electronically focusing arrays.

FIG. 16 depicts on the left a low-resolution image (first ultrasound image 412). The area within the rectangle 1602 is the area chosen for the high-resolution image (second ultrasound image 442) in the middle. On the right the crop taken out of the liquid area of the second ultrasound image 442 is shown (cropped image 1610), which is used for the final cell-count model.

The images are preferably preprocessed in the same manner as the low-resolution images (e.g. Butterworth bandpass filter of 6^{th} order between 15 and 25 MHz; Hilbert transform and signal envelopes; adjusting dynamic range between a minimum and a maximum value, applying a min-max normalization). Then, before applying the final cell detection and count models on the preprocessed high-resolution images, another quality check model may be applied to ensure that the images still fulfil the quality criteria checked for in low-resolution images and in general to ensure that the images are of sufficient quality and are focused on the right area. If this is not the case, low-resolution mode is applied again to find a better area or a solution to the problem at hand.

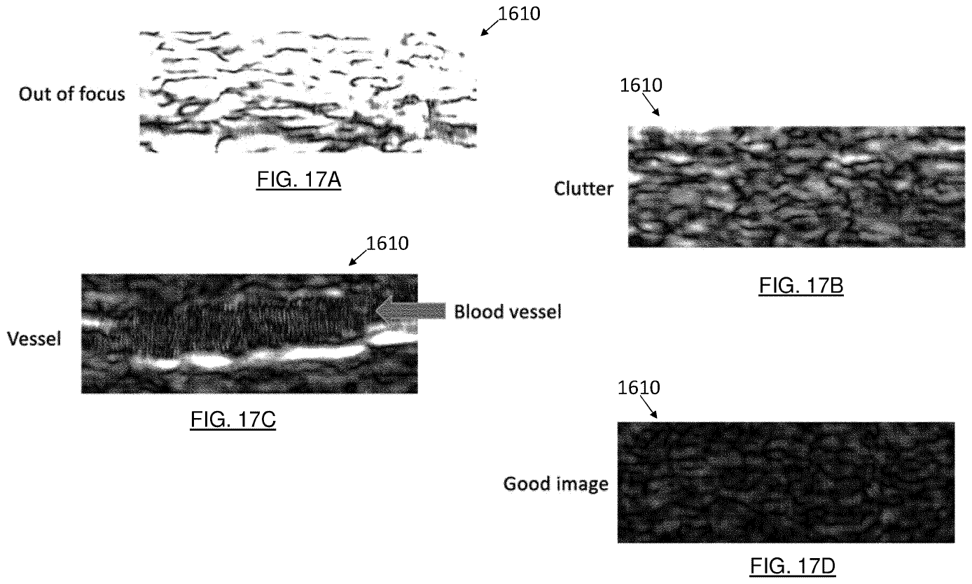

In the CSF use case where high-resolution images of the transfontanellar liquid space are acquired, a deep learning model is trained to automatically classify the images into predetermined classes, such as: good image, out of focus, clutter and vessel. Different number and type of classes may be used. The model takes as input a single image and outputs a probability for being in each of the classes. The maximum value of these four classes is taken to decide which class the image should be classified into.
The class “Out of focus” consists of images where the focused area in high-resolution mode was not the correct one, or the device or patient moved since the best focus area was detected and, consequently, the region for zooming into high-resolution needs to be redefined in low-resolution mode (FIG. 17A).
“Clutter” means that there is too much noise present in the image, either because of bad coupling or because of noise caused by the tissue above the liquid area (FIG. 17B). This problem can be solved by repositioning the ultrasound probe or by improving the acoustic coupling with tissue. It implies circling back to low-resolution mode.
“Vessel” is a specific problem of the CSF use case caused by blood vessels passing through the liquid area in the extra-axial space below the fontanel tissue. The blood vessels make it impossible to apply a predictive model for cell counting, since blood vessels contain both red and white blood cells and do not represent the cell concentration in the fluid. These images need to be discarded and cannot be used for the cell count models (FIG. 17C). To solve this problem, low-resolution mode is applied again to find a more adequate area.
“Good image”: An example of a good quality high-resolution cropped image 1610 is shown in FIG. 17D.

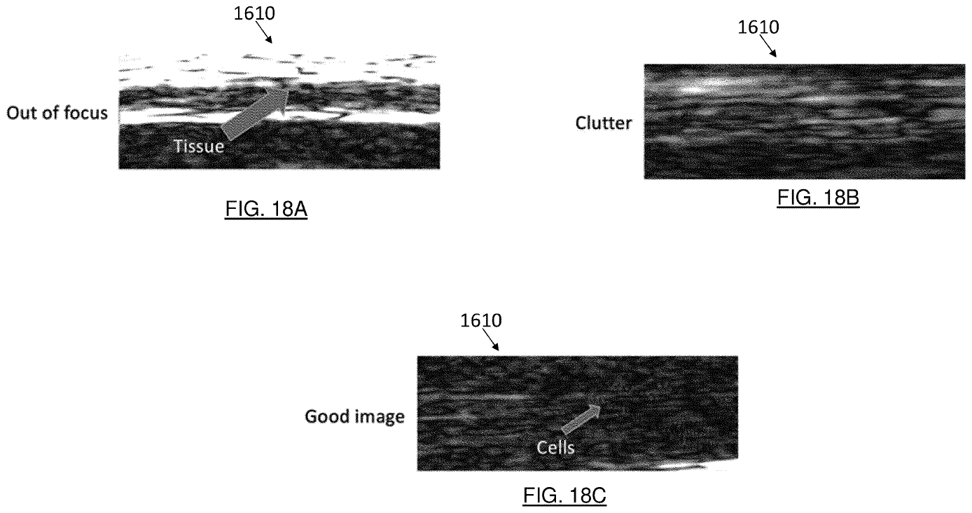

In an embodiment applied to the use case of high-resolution images of the aqueous humour, the same type of model as in the CSF use case is applied, with the only difference that the class “vessel” is not used. FIG. 18A shows an “Out of focus” image example, wherein the image was cropped in the wrong area (likely due to movement of the probe or patient). FIG. 18B shows a “Clutter” image example, since too much clutter is present in this image. FIG. 18C shows a “Good image” example (this image crop is valid for being used for the cell-counting model).

In the case of high-resolution images of peritoneal liquid in peritoneal dialysis bags 109 no quality model is really needed, since the setup is fixed and the quality detection model applied in low-resolution is often sufficient in this case.

The quality detection model for high-resolution images follows the same procedure in all cases. The only difference between different applications is the addition or the removal of one of the classes, but the model can be summarized-like in the low-resolution quality control model-to good image vs. bad image, where the bad image class may contain several sub-classes (e.g. “Clutter”, “Out of focus”).

As shown in the flowchart of FIG. 19, the method 400 may therefore comprise preprocessing 1920 the second ultrasound image 442 (or the cropped image 1610, when the method comprises cropping 1910 the second ultrasound image 442 around the target focal point) to obtain a preprocessed image 1912 prior to predicting 450 a concentration of white blood cells 130, wherein the preprocessing may include applying a bandpass filter, a Hilbert transform, a min-max normalization, an image size reduction or a combination thereof. The method 400 may also comprise classifying 1930 the quality of said preprocessed image 1912 using an artificial neural network trained with manually labelled preprocessed images, and proceed to predicting 450 a concentration of white blood cells 130 only when the preprocessed image is classified into a predetermined class or classes (e.g. “Good image”). In the example, it is checked 1940 whether the preprocessed image 1912 is classified as “Good image” (i.e. good quality image); in that case the process continues with the next step 450, otherwise (or, alternatively, when a determine number—e.g. five—of consecutive second ultrasound images 440 are classified as “Bad image”) the process starts again by generating 410 and analysing a new fist ultrasound image 412 acquired at lower spatial resolution.

The next and last step of the method 400 is predicting 450 a concentration of white blood cells 130 in the second ultrasound image 442 (in particular, in the preprocessed image 1912 in the embodiment of FIG. 19) using an artificial neural network trained with images with an assigned ground truth concentration. White blood cells presence detection and cell counting at image frame level. As for the detection of the presence of white blood cells in the focal area of high-resolution ultrasound images, the count can be performed based on different strategies. The common ground of all deep learning models applied on this type of images is the assignment of the ground truth of the true number of white blood cells present in the liquid based on the clinical assessment of the patients. Based on this, the high-resolution pre-processed images 1912 are assigned a ground truth concentration which is used for training the deep learning models, which then, consequently, assign this prediction of the ground truth automatically to new images. Two different strategies are considered:
1. Classification algorithms, which classify the images into certain ranges of numbers of white blood cells. These ranges represent clinically important thresholds, such as classifying images into either healthy or disease classes (presence of a certain amount of white blood cells) and then subsequently refining the disease class into finer classes with different ranges of number of white blood cells, to obtain information about the severity of the disease (the higher the number of white blood cells, the more severe the disease). In the case of a classification algorithm per numbers of white blood cell ranges, the input into the network is a single high-resolution image frame and the output is a probability of being in a specific class associated to a range of numbers of cells.
2. A convolutional neural network algorithm, which, instead of having as output a probability of being in a specific class representing a range of numbers, provides the number of white blood cells present. In this case, the algorithm is trained in such a way, that each image is associated to a specific number of white blood cells, known from the ground truth, and thus learns this association. The output is a regression model giving an estimation of the exact number of white blood cells present in the image, which is also able to interpolate the values.

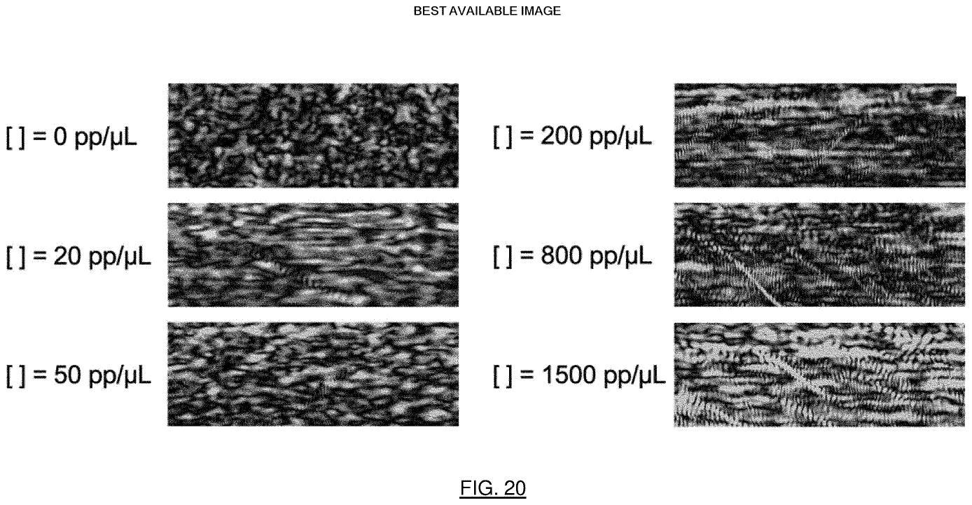

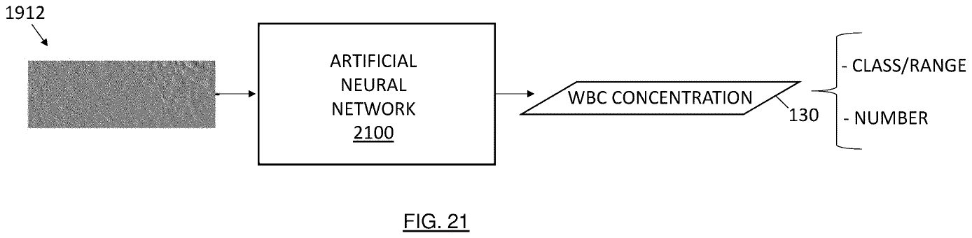

In these models for cell detection and cell counting there is no difference between different use cases. FIG. 20 shows images with different concentrations of amounts of cells present in the serous fluid area. In vitro recorded images are shown based on polystyrene particles mimicking white blood cells, wherein the traces produced by the particle movement the liquid can be observed. The ground truth for in vivo data can be obtained, for instance, through lumbar puncture and a further analysis of white blood cells in the cerebrospinal fluid, according to the common practice. In the case of in vitro data, the ground truth the concentrations are created in laboratory. The models are trained with the ground truth, using a number, range of values or class. The training of a neural network is done as customary, by reducing the prediction error based on the ground truth.

FIG. 21 shows an artificial neural network 2100, such as a convolutional neural network, used to predict a concentration of white blood cells 130 in the high-resolution preprocessed image 1912 trained with images with an assigned ground truth concentration. As explained above, the output of the artificial neural network 2100 (WBC concentration 130) may be, for instance, an estimation of the number (e.g. 53 pp/μL) of white blood cells concentration using a regression model, or a class (e.g high concentration of WBC, low concentration of WBC) using a classification model representing a range (e.g. 50-200 pp/μL) of white blood cells concentration. The input of the artificial neural network 2100 may be a plurality of high-resolution preprocessed images 1912 (or second ultrasound images 442), instead of the single input image shown in FIG. 21.

The concentration of white blood cells 130 may be predicted based on a plurality of preprocessed images 1912 and/or their corresponding classification/regression, instead of just one image. Based on these models, the overall cell count for a patient can be calculated based on a collection of several image frames recorded from the same patient. The final number may be estimated by either applying a soft voting technique, which estimates a final prediction over several single predictions.

In the case of classification by ranges, this may be done either by calculating a weighted average over the probabilities of being in each class over all image frames for one patient, giving a final probability value per class. The maximum value of these final probability values indicates then the class the patient should be classified into. Another possibility is to calculate the median over probabilities and perform the same procedure for deciding on the final class association.

In the regression case, the same process is applied, but instead of calculating a weighted average or the median over probabilities of belonging to a specific class, these metrics are directly calculated over the estimates of the number of white blood cells.

FIG. 22 shows a scheme of calculating the final decision (WBC concentration 130) per patient: the model decision (using an artificial neural network 2100 as a classifier or regressor) is computed for each image frame (preprocessed image 1912), obtaining a prediction (P₁, P₂, P₃, P₄, . . . , P_{N}) for each image and then a final decision is calculated per patient as a function of the predictions F(P₁, P₂, P₃, P₄, . . . , P_{N}), e.g. by taking the median or a weighted average over the single outcomes. The computation of the final decision per patient, be it with a classification model or with a regression model, is not depending on any specific application or use case.

In an embodiment, the present invention further analyzes the white blood cells present in serous fluids to automatically detect the different types of white blood cells in order to discriminate the types of infections. With different pathogens, different types of white blood cells prevail. In meningitis, for example, mainly two types of white blood cells are present in the cerebrospinal fluid, lymphocytes and neutrophils, where in viral meningitis mainly lymphocytes are present and in bacterial infection mainly neutrophils are present [7]. These two types of white blood cells differ, among other things, in their size, the lymphocytes being smaller than the neutrophils.

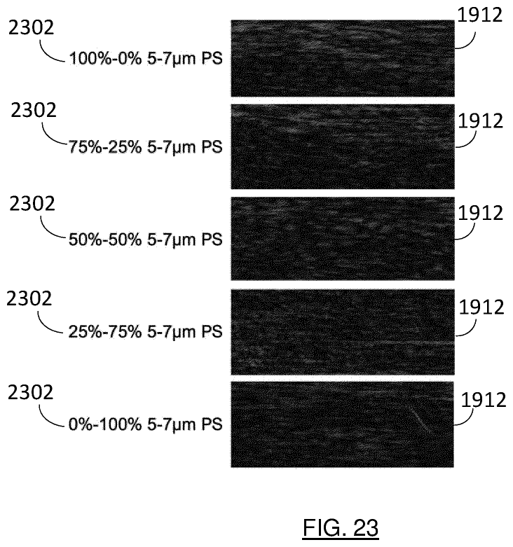

For distinguishing between different compositions of different types of white blood cells, which differ in size, a deep learning classification model is trained based on in vitro data recorded in a laboratory setup, with several classes (e.g. 5 classes) of different compositions of particles mimicking the sizes of lymphocytes and neutrophils, ranging from 100% lymphocyte sized particles to 100% of neutrophil sized particles with mixtures of both particles in the middle (25% of one type and 75% of the other type or 50%-50%). FIG. 23 shows high-resolution preprocessed cropped images 1912 with different compositions of particles having sizes of 5 μm and 7 μm, respectively mimicking the backscatter signal reflected by lymphocytes and neutrophils. In particular, from top to bottom, the following 5 classes are considered: 100% lymphocyte mimicking particles (5 μm)-0% neutrophils mimicking particles (7 μm); 75% lymphocyte mimicking particles (5 μm)-25 % neutrophils mimicking particles (7 μm); 50% lymphocyte mimicking particles (5 μm)-50 % neutrophils mimicking particles (7 μm); 25% lymphocyte mimicking particles (5 μm)-75 % neutrophils mimicking particles (7 μm); 0% lymphocyte mimicking particles (5 μm)-100 % neutrophils mimicking particles (7 μm).

The methodology applied follows the same logic as in the above-described models, where high-resolution images (e.g. preprocess images 1912) are assigned one of the predetermined classes 2302 and thus trained with this ground truth information. The input of the final model is the focal area of a pre-processed high-resolution image and the output is a probability for this image of belonging to each of the classes. As above, a global probability may be calculated for a whole subject based on the mean or median over probabilities and thus a final probability for a subject for each class is computed. The class with the highest probability value is then chosen as the final output class. With this methodology amounts of different types of white blood cells can be detected with high-resolution ultrasound images of serous fluids in a non-invasive manner.

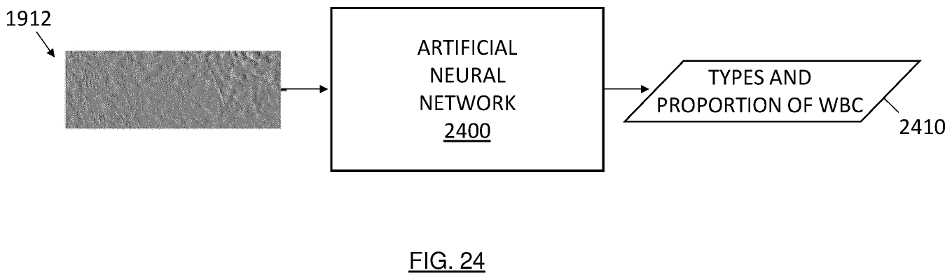

FIG. 24 represents a neural artificial network 2400 which is trained with images with an assigned ground truth corresponding to classes 2302 associated to different proportions of different types of WBC, as depicted in the example of FIG. 23. The output of the so-trained neural artificial network 2400 is the types and proportion of WBC 2410 detected, which may be a class 2302 representing the types of WBC present in the liquid and their proportions (e.g. 75% lymphocytes-25% neutrophils). The input of the artificial neural network 2400 may be a plurality of high-resolution preprocessed images 1912 (or second ultrasound images 442), instead of the single input image shown in FIG. 24.

The image processing unit 120 may be configured to predict a group of pathogens causing an infection from seriated measurements of types and proportion of white blood cells 2410 obtained each of them from at least one second ultrasound image 442 (or a preprocessed image 1912), using for instance an artificial neural network previously trained. Similarly, the method may also comprise predicting a group of pathogens causing an infection from seriated measurements of types and proportion of white blood cells 2410 obtained each of them from at least one second ultrasound image 442.

By combining all above-described models the system and method of the present invention can count white blood cells non-invasively, overcoming several technical difficulties, such as the automatic exclusion of low-quality images, the automatic finding of the optimal region to measure the amount of white blood cells in the body fluid and the detection of blood vessels and other different anatomical structures in images, like different types of tissue. The invention combines different artificial intelligence models, based majorly on deep convolutional network architecture. There is no existing technique able to count white blood cells in body fluids in a completely non-invasive and automatized manner, and thus able to detect inflammatory diseases and provide information about the severity of the disease, depending on the number of white blood cells present and the composition of different types thereof. Furthermore, frequent measurements of the white blood cell count in an infected serous fluid informs about the response to treatment of the patient. Rapid and positive responses to treatment can be related to rapidly decaying WBC counts. Likewise, positive but delayed responses to treatment can be related to decaying WBC counts at a smaller slope. And non-responding patients have a very small slope because the number of WBC decays very mildly, if it does even decay. Interestingly, WBC decay patterns can be related to groups of pathogens causing the infection [8].

1. A system for non-invasive white blood cell counting in serous body fluids, the system comprising:
an ultrasound device configured to generate ultrasound images from a body or a sample of serous body fluid at a configurable spatial resolution in a scan direction; and
an image processing unit configured to:
receive at least one first ultrasound image generated by the ultrasound device at a first spatial resolution in the scan direction;
for each first ultrasound image:
determine a region corresponding to a serous body fluid in the first ultrasound image;
determine a target focal point in the region; and
receive at least one second ultrasound image generated by the ultrasound device focused on the target focal point at a second spatial resolution in the scan direction, higher than the first spatial resolution; and
predict a concentration of white blood cells from the at least one second ultrasound image using an artificial neural network trained with images with an assigned ground truth concentration.

2. The system of claim 1, wherein the image processing unit is configured to determine a region corresponding to a serous body fluid by segmenting a region in the first ultrasound image using an artificial neural network trained with images including manually segmented regions.

3. The system of claim 1, wherein the image processing unit is configured to crop each second ultrasound image around the target focal point and predict the concentration of white blood cells from at least one cropped image.

4. The system of claim 1, wherein the image processing unit is configured to preprocess each first ultrasound image or second ultrasound image to obtain a preprocessed image.

5. The system of claim 4, wherein the image processing unit is configured to preprocess each first ultrasound image or second ultrasound image by applying a bandpass filter, a Hilbert transform and a min-max normalization.

6. The system of claim 4, wherein the image processing unit is configured to classify the quality of each preprocessed image using an artificial neural network trained with manually labelled preprocessed images, and check whether the preprocessed image is classified into a predetermined class.

7. The system of claim 1, wherein the ultrasound device comprises an ultrasound transducer and a motor configured to displace the ultrasound transducer along a longitudinal axis to change the focal depth; and
wherein the image processing unit is configured to activate the motor, via a focus positioning control signal, so that the ultrasound device is focused on the target focal point prior to generate the at least one second ultrasound image.

8. The system of claim 1, wherein the image processing unit is configured to predict the types and proportion of white blood cells from at least one second ultrasound image using an artificial neural network trained with images with an assigned ground truth.

9. The system of claim 1, wherein the image processing unit is configured to focus the ultrasound device on the target focal point determined for each first ultrasound image.

10. A method of non-invasive white blood cell counting in serous body fluids, the method comprising:
generating, by an ultrasound device, at least one first ultrasound image from a body or a sample of serous body fluid at a first spatial resolution in the scan direction;
for each first ultrasound image;
determining a region corresponding to a serous body fluid in the first ultrasound image;
determining a target focal point in the region;
focusing the ultrasound device on the target focal point; and
generating, by the ultrasound device, at least one second ultrasound image from the body or the sample at a second spatial resolution in the scan direction, higher than the first spatial resolution; and
predicting a concentration of white blood cells from the at least one second ultrasound image using an artificial neural network trained with images with an assigned ground truth concentration.

11. The method of claim 10, further comprising preprocessing each first ultrasound image or second ultrasound image to obtain a preprocessed image.

12. The method of claim 11, wherein preprocessing each first ultrasound image or second ultrasound image includes applying a bandpass filter, a Hilbert transform and a min-max normalization.

13. The method of claim 11, further comprising classifying the quality of each preprocessed image using an artificial neural network trained with manually labelled preprocessed images, and checking whether the preprocessed image is classified into a predetermined class.

14. The method of claim 10, further comprising predicting the types and proportion of white blood cells from at least one second ultrasound image using an artificial neural network trained with images with an assigned ground truth.

15. The method of claim 14, further comprising predicting a group of pathogens causing an infection from seriated measurements of types and proportion of white blood cells obtained each of them from at least one second ultrasound image.

16. A non-transitory computer-readable storage medium for non-invasive white blood cell counting in serous body fluids, comprising computer code instructions that, when executed by a processor, causes the processor to perform the method of claim 10.
